SEQUENCE LISTING (14 sequences)

SEQ ID NO: 1 - protein, 271 aa, synthetic construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYODLAILWNCLGEFSPSLOKRLFOKYGIDNPDMNKLOFHLMLDEFF

SEQ ID NO: 2 - protein, 396 aa, synthetic construct
MKSNNALIVILGTVTLDAVGIGLVMPVLPGLLRDIVHSDSIASHYGVLLALYALMQFLCAPVLGALSDRFGRRPVLLASLLGATIDYAIMATTPVLWILYAGRIVAGITGATGAVAGAYIADITDGEDRARHFGLMSACFGVGMVAGPVAGGLLGAISLHAPFLAAAVLNGLNLLLGCFLMQESHKGERRPMPLRAFNPVSSFRWARGMTIVAALMTVFFIMQLVGQVPAALWVIFGEDRFRWSATMIGLSLAVFGILHALAQAFVTGPATKRFGEKQAIIAGMAADALGYVLLAFATRGWMAFPIMILLASGGIGMPALQAMLSRQVDDDHQGQLQGSLAALISLTSITGPLIVTAIYAASASTWNGLAWIVGAALYLVCLPALRRGAWSRATST

SEQ ID NO: 3 - protein, 473 aa, synthetic construct
MNIKKFAKQATVLTFTTALLAGGATQAFAKETNQKPYKETYGISHITRHDMLQIPEQQKNEKYQVPEFDSSTIKNISSAKGLDVWDSWPLQNADGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASDSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 4 - protein, 1024 aa, synthetic construct
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 5 - DNA, 446 bp, synthetic construct
  source 1..446: mol_type = other DNA
cacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacg

SEQ ID NO: 6 - DNA, 76 bp, synthetic construct
  source 1..76: mol_type = other DNA
ttgacagcttatcatcgataaactgtaatgcggtagtttatcacagttaaattgctaacgcagtcaggcaccgtgt

SEQ ID NO: 7 - DNA, 101 bp, synthetic construct
  source 1..101: mol_type = other DNA
ttgtgtctcaaaatctctgatgttacattgcacaagataaaaatatatcatcatgaacaataaaactgtctgcttacataaacagtaatacaaggggtgtt

SEQ ID NO: 8 - DNA, 67 bp, synthetic construct
  source 1..67: mol_type = other DNA
gagctgttgacaattaatcatcggctcgtataatgtgtggaattgtgagcggataacaatttcacac

SEQ ID NO: 9 - DNA, 64 bp, synthetic construct
  source 1..64: mol_type = other DNA
ttgacggctagctcagtcctaggtacagtgctagccaagaagctttattttggggagacacatc

SEQ ID NO: 10 - DNA, 112 bp, synthetic construct
  source 1..112: mol_type = other DNA
cagctgtctagggcggcggatttgtcctactcaggagagcgttcaccgacaaacaacagataaaacgaaaggcccagtctttcgactgagcctttcgttttatttgatgcct

SEQ ID NO: 11 - DNA, 103 bp, synthetic construct
  source 1..103: mol_type = other DNA
cttggactcctgttgatagatccagtaatgacctcagaactccatctggatttgttcagaacgctcggttgccgccgggcgttttttattggtgagaatccag

SEQ ID NO: 12 - DNA, 246 bp, synthetic construct
  source 1..246: mol_type = other DNA
cttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgta

SEQ ID NO: 13 - DNA, 392 bp, synthetic construct
  source 1..392: mol_type = other DNA
gatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatgg

SEQ ID NO: 14 - protein, 417 aa, synthetic construct [sequence omitted]

(19) United States
(12) Patent Application Publication
(10) Pub. No.: US 2026/0337619 A1
(43) Pub. Date: Oct. 29, 2026

(54) COMPOSITIONS AND METHODS FOR ENGINEERED BACTERIA

(71) Applicants: Nitto Denko Corporation, Osaka (JP); Safion Renewables Inc., Cos Cob, CT (US)

(72) Inventors: Andrew John BORCHERT, Arvada, CO (US); Lauren Ann Riley DRUMM, Lafayette, CO (US)

(21) Appl. No.: 19/328,010

(22) Filed: Sep. 12, 2025

Related U.S. Application Data
(60) Provisional application No. 63/714,726, filed on Oct. 31, 2024.

(51) Int. Cl. C12N 15/74; C12N 9/10; C12N 9/80

(52) U.S. Cl. CPC C12N 15/74; C12N 9/1048; C12N 9/80; C12N 2800/60; C12Y 305/01097

(57) ABSTRACT

Provided herein are compositions, systems, and methods for genetically modifying an organism and selecting a modified organism.

### CROSS REFERENCE TO RELATED APPLICATIONS

This application claims the benefit of U.S. Provisional Application No. 63/714,726, filed Oct. 31, 2024.

### REFERENCE TO SEQUENCE LISTING

The present application is being filed along with a Sequence Listing in electronic format. The Sequence Listing is provided as a file entitled NDTUS.010A.xml, which was created and last modified on Nov. 4, 2025, and is 17,313 bytes in size. The information in the electronic Sequence Listing is hereby incorporated by reference in its entirety.

### BACKGROUND

Organisms can be engineered or modified to improve production of bioproducts. Engineering techniques include genetically modifying an organism. Engineering biotechnologically relevant bacteria may involve disruption of genes or introduction of new nucleotide sequences into the relevant organism. Techniques and selection/counter-selection-based systems may enable identification of organisms comprising a desired modification. Adaptable platforms can be used for engineering genomes of many types of organisms.

### SUMMARY

Provided herein, in some embodiments, are compositions, systems, and methods for producing and selecting a modified organism. The methods provided herein can be alternatives to standard processes for genetic modification of organisms, allowing for more rapid, less costly, more efficient mutant strain construction compared to other tools and methods for engineering organisms.

Provided herein, in some embodiments, is a vector comprising a first nucleic acid sequence comprising a positive selection marker gene, a second nucleic acid sequence comprising a first counter-selection marker gene, a third nucleic acid sequence comprising a second counter-selection marker gene, and a fourth nucleic acid sequence comprising a reporter gene.

In some embodiments, the vector comprises a fifth nucleic acid sequence comprising a first homology arm that comprises homology with a first portion of a target genomic sequence of an organism targeted for modification. In some embodiments, the vector comprises a sixth nucleic acid sequence comprising a second homology arm that comprises homology with a second portion of the target genomic sequence of the organism targeted for modification. In some embodiments, the vector comprises a genetic modification sequence flanked by the first homology sequence and the second homology sequence.

In some embodiments, the positive selection marker gene encodes aminoglycoside phosphotransferase, a class A tetracycline resistance protein, chloramphenicol acetyltransferase, or beta-lactamase. In some embodiments, the positive selection marker gene is involved in the metabolism of amino acids, carbohydrates, lipids, nucleotides, or vitamins. In some embodiments, the positive selection marker gene encodes orotate phosphoribosyl-transferase (pyrE) galactokinase 1 (galK), thymidylate synthase (thyA), orotidine-5′-monophosphate decarboxylase (pyrF), or uracil phosphoribosyltransferase (upp). In some embodiments, the vector comprises a second positive selection marker gene.

In some embodiments, the first counter-selection marker gene encodes levansucrase, a class A tetracycline resistance protein, uracil phosphoribosyl-transferase, orotate phosphoribosyl-transferase, or cytosine deaminase. In some embodiments, the second counter-selection marker gene encodes levansucrase, a class A tetracycline resistance protein, uracil phosphoribosyl-transferase, orotate phosphoribosyl-transferase, or cytosine deaminase.

In some embodiments, the reporter gene comprises a visual reporter gene. In some embodiments, the reporter gene comprises lacZ, a lacZ homolog, a gene encoding green fluorescent protein (GFP), a gene encoding red fluorescent protein (RFP), or a gene encoding luciferase (luc).

In some embodiments, the genetic modification sequence comprises a sequence of interest.

In some embodiments, any of the first, second, third, fourth, fifth, or sixth nucleic acid sequences are operably linked to a regulatory element. In some embodiments, the vector further comprises one or more of a promoter, a ribosomal binding site (RBS), a transcription terminator, an origin of transference (OriT), an origin of replication (Ori), a multiple cloning site (MCS), or a transporter. In some embodiments, the regulatory element comprises the promoter. In some embodiments, the promoter comprises a native promoter, a hybrid promoter, or a synthetic promoter designed in silico. In some embodiments, the native promoter comprises a native promoter for sacB, a native promoter for tetA, or a native promoter for aphA1. In some embodiments, the hybrid promoter comprises a tac promoter. In some embodiments, the synthetic promoter comprises a BBa_J23100 promoter. In some embodiments, the RBS comprises a nucleotide sequence derived from a Gram-negative bacteria or a synthetic RBS designed in silico. In some embodiments, the transcription terminator comprises an rrnB terminator, a rho-independent terminator of phage λ, a nucleotide sequence derived from AAB, a nucleotide sequence derived from a Gram-negative bacteria, or a transcriptional terminator sequence designed in silico. In some embodiments, the OriT enables conjugative transfer of nucleic acids between bacterial cells. In some embodiments, the OriTis derived from an RP4 plasmid. In some embodiments, the Ori enables vector replication in a microorganism used to store, modify, or produce the vector. In some embodiments, the Ori is unable to facilitate replication of the vector in a genetic background subject to genomic modification. In some embodiments, the Ori is R6Kγ. In some embodiments, the MCS encodes at least two restriction enzyme recognition sites. In some embodiments, the at least two restriction enzyme recognition sites comprise restriction enzyme recognition sites for SacI, SmaI, BamHI, HindIII, XbaI, SaII, PstI, or SphI. In some embodiments, the transporter is lacY.

Provided herein, in some embodiments, is a system for genetic modification of an organism comprising the vector provided herein.

In some embodiments, the positive selection marker gene (i) confers resistance to an antibiotic or (ii) creates a cell survival molecule. In some embodiments, the second positive selection marker gene (i) confers resistance to an antibiotic or (ii) creates a cell survival molecule. In some embodiments, the antibiotic is kanamycin, tetracycline, chloramphenicol, ampicillin, carbenicillin, gentamicin, or erythromycin. In some embodiments, the cell survival molecule is involved in the metabolism of amino acids, carbohydrates, lipids, nucleotides, or vitamins. In some embodiments, the cell survival molecule is orotate phosphoribosyl-transferase, galactokinase 1, thymidylate synthase, orotidine-5′-monophosphate decarboxylase, or uracil phosphoribosyltransferase. In some embodiments, the second positive selection marker gene improves the genetic modification in the organism when the organism exhibits natural resistance to the antibiotic or when the organism natively expresses the cell survival molecule.

In some embodiments, the first counter-selection marker gene inhibits or eliminates growth of the organism when a first counter-selection condition is present. In some embodiments, the second counter-selection marker gene inhibits or eliminates growth of the organism when a second counter-selection condition is present.

In some embodiments, the reporter gene generates a visual phenotype.

In some embodiments, the first homology arm and the second homology arm allow integration of the vector into the target genomic sequence. In some embodiments, the first homology arm and the second homology arm allow excision of a backbone of the vector from the target genomic sequence after the integration.

In some embodiments, the presence of the first counter-selection condition reduces the possibility that a frameshift mutation, a missense mutation, or a nonsense mutation in either the first counter-selection marker gene or the second counter-selection marker gene allows for growth of the organism without the excision. In some embodiments, the presence of the second counter-selection condition reduces the possibility that a frameshift mutation, a missense mutation, or a nonsense mutation in either the first counter-selection marker gene or the second counter-selection marker gene allows for growth of the organism without the excision.

In some embodiments, the genetic modification sequence causes insertion, enhancement, deletion, or mitigation of a sequence of interest. In some embodiments, the genetic modification sequence causes insertion, deletion, or substitution of a single nucleotide; insertion, deletion, or substitution of two or more nucleotides; or two or more non-contiguous insertions, deletions, or substitutions.

In some embodiments, the organism is a Gram-negative bacterium. In some embodiments, the organism is a Zymomonas bacterium, a Pseudomonas bacterium, a Lactobacillus bacterium, a Escherichia bacterium, or an Acinetobacter bacterium.

Provided herein, in some embodiments, is a method for modifying an organism comprising introducing the vector provided herein or the system provided herein to the organism. In some embodiments, introducing the vector or the system to the organism causes a genetic modification in the organism to produce a modified organism.

Provided herein, in some embodiments, is a method for selecting a modified organism. In some embodiments, the method comprises (a) introducing the vector provided herein or the system provided herein to a first population of cells. In some embodiments, introducing the vector or the system to the first population generates a second population of cells. In some embodiments, the vector is integrated into a target genome of the second population. In some embodiments, the method comprises (b) growing the first population and the second population in a first media comprising a positive selection agent and a visual screening agent. In some embodiments, the method comprises (c) selecting the second population based on a visual phenotype. In some embodiments, the method comprises (d) growing the second population in a second media comprising a first counter-selection condition, a second counter-selection condition, and the visual screening agent. In some embodiments, growing the second population in the second media generates a third population of cells. In some embodiments, a backbone of the vector is excised from a target genome of the third population. In some embodiments, the third population comprises the modified organism. In some embodiments, the method comprises (e) selecting the third population based on the visual phenotype.

Provided herein, in some embodiments, is a method for selecting a modified organism. In some embodiments, the method comprises (a) introducing a vector to a first population of cells. In some embodiments, introducing the vector to the first population generates a second population of cells. In some embodiments, the vector is integrated into a target genome of the second population. In some embodiments, the method comprises (b) growing the first population and the second population in a first media comprising a positive selection agent and a visual screening agent. In some embodiments, the method comprises (c) selecting the second population based on a visual phenotype. In some embodiments, the method comprises (d) growing the second population in a second media comprising a first counter-selection condition, a second counter-selection condition, and the visual screening agent. In some embodiments, growing the second population in the second media generates a third population of cells. In some embodiments, a backbone of the vector is excised from a target genome of the third population. In some embodiments, the third population comprises the modified organism. In some embodiments, the method comprises (e) selecting the third population based on the visual phenotype.

In some embodiments, the method comprises (f) screening the selected third population for a genetic modification sequence.

In some embodiments, the vector comprises a first nucleic acid sequence comprising a positive selection marker gene, a second nucleic acid sequence comprising a first counter-selection marker gene, a third nucleic acid sequence comprising a second counter-selection marker gene, and a fourth nucleic acid sequence comprising a reporter gene. In some embodiments, the vector comprises a fifth nucleic acid sequence comprising a first homology arm that comprises homology with a first portion of the target genome of the second population, a sixth nucleic acid sequence comprising a second homology arm that comprises homology with a second portion of the target genome of the second population, and a genetic modification sequence flanked by the first homology sequence and the second homology sequence.

In some embodiments, introducing the vector to the first population is via electroporation, conjugation, transduction, or transfection. In some embodiments, integration of the vector into the target genome of the second population introduces the genetic modification sequence to the target genome of the second population. In some embodiments, integration of the vector into the target genome of the second population is via homologous recombination. In some embodiments, the integration is via endogenous homologous recombination machinery. In some embodiments, the integration is via heterologous homologous recombination machinery.

In some embodiments, the positive selection agent (i) permits growth of the second population and (ii) prevents growth of the first population. In some embodiments, the positive selection agent permits growth when the positive selection marker gene is present. In some embodiments, the positive selection agent prevents growth when the positive selection marker gene is not present.

In some embodiments, the visual screening agent (i) elicits the visual phenotype in the second population and (ii) does not elicit the visual phenotype in the first population. In some embodiments, the visual screening agent (i) elicits the visual phenotype in the second population and (iii) does not elicit the visual phenotype in the third population. In some embodiments, the visual screening agent elicits the visual phenotype when the reporter gene is present. In some embodiments, the visual screening agent does not elicit the visual phenotype when the reporter gene is not present. In some embodiments, the selecting the second population is based on presence of the visual phenotype. In some embodiments, the selecting the second population is based on absence of the visual phenotype.

In some embodiments, the second media does not comprise the positive selection agent.

In some embodiments, the first counter-selection condition (i) permits growth of the third population and (ii) prevents growth of the second population. In some embodiments, the first counter-selection condition permits growth of the first population and the third population. In some embodiments, the first counter-selection condition prevents growth of the second population. In some embodiments, the first counter-selection condition permits growth when the first counter-selection marker gene is present. In some embodiments, the first counter-selection condition prevents growth when the first counter-selection marker gene is not present.

In some embodiments, the second counter-selection condition (i) permits growth of the third population and (ii) prevents growth of the second population. In some embodiments, the second counter-selection condition permits growth when the second counter-selection marker gene is present. In some embodiments, the second counter-selection condition prevents growth when the second counter-selection marker gene is not present. In some embodiments, the second counter-selection condition permits growth of the first population and the third population. In some embodiments, the second counter-selection condition prevents growth of the second population.

In some embodiments, excision of the vector backbone from the target genome of the third population removes the vector backbone from the target genome of third second population. In some embodiments, the target genome of the third population retains the genetic modification sequence upon the excision. In some embodiments, the excision is via homologous recombination. In some embodiments, the excision is via heterologous homologous recombination machinery.

In some embodiments, the genetic modification sequence knocks out, disrupts, knocks down, or inhibits activity of at least one sequence of interest or at least one gene product in the target genome of the third population. In some embodiments, the genetic modification sequence enhances expression or activity of at least one sequence of interest or at least one gene product in the target genome of the third population. In some embodiments, the genetic modification sequence introduces at least one sequence of interest to the target genome of the third population. In some embodiments, the vector produces a scarless and stable genetic modification in the target genome of the third population.

In some embodiments, the selecting the third population is based on presence of the visual phenotype. In some embodiments, the selecting the third population is based on absence of the visual phenotype.

In some embodiments, the modified organism is an acetic acid bacterium.

In some embodiments, introducing the vector produces fewer false-positives than introducing a vector that does not comprise the positive selection marker gene, the first counter-selection marker gene, the second counter-selection marker gene, or the reporter gene.

Provided herein, in some embodiments, is a method for selecting a modified organism. In some embodiments, the method comprises (a) introducing a vector to a first population of cells. In some embodiments, introducing the vector to the first population generates a second population of cells. In some embodiments, the vector is integrated into a target genome of the second population. In some embodiments, the vector introduces a genetic modification sequence to the target genome of the second population. In some embodiments, the method comprises (b) growing the first population and the second population in a first media comprising a positive selection agent and a visual screening agent. In some embodiments, the positive selection agent (i) permits growth of the second population and (ii) prevents growth of the first population. In some embodiments, the visual screening agent (i) elicits a visual phenotype in the second population and (ii) does not elicit the visual phenotype in the first population. In some embodiments, the method comprises (c) selecting the second population based on the visual phenotype. In some embodiments, the method comprises (d) growing the second population in a second media. In some embodiments, the second media does not comprise the positive selection agent. In some embodiments, the second media comprises a first counter-selection condition, a second counter-selection condition, and the visual screening agent. In some embodiments, the first counter-selection condition (i) permits growth of the third population and (ii) prevents growth of the second population. In some embodiments, the second counter-selection condition (i) permits growth of the third population and (ii) prevents growth of the second population. In some embodiments, the visual screening agent (i) elicits the visual phenotype in the second population and (iii) does not elicit the visual phenotype in the third population. In some embodiments, growing the second population in the second media generates a third population of cells. In some embodiments, a backbone of the vector is excised from a target genome of the third population. In some embodiments, the target genome of the third population comprises the genetic modification sequence. In some embodiments, the third population comprises the modified organism. In some embodiments, the method comprises (e) selecting the third population based on the visual phenotype. In some embodiments, the method comprises (f) screening the selected third population for the genetic modification sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A shows an illustrative minimal vector comprising a positive selection marker gene, a first counter-selection marker, a second counter-selection marker, a reporter gene, a homology region comprising a first homology arm and a second homology arm, and a genetic modification sequence (e.g., “Desired Mutation”).

FIG. 1B shows an illustrative vector comprising a positive selection marker gene (e.g., aphA1), a first counter-selection marker (e.g., tetA), a second counter-selection marker (e.g., sacB), a reporter gene (e.g., lacZ for blue/white visual screening with X-Gal), a lactose permease (e.g., lacY for X-Gal transport), a homology region comprising a first homology arm and a second homology arm (comprising sequences upstream and downstream of the wcaE coding sequence), and a genetic modification sequence (comprising a truncated wcaE coding sequence (“wcaE CDS”)). In some embodiments, the vector comprises an OriT (e.g., RP4 OriT), an Ori (e.g., R6K Ori), promoter sequences (e.g., pTetA, BBa_pJ23100, pAphA1, pSacB), RBS sequences, and terminator sequences (e.g., rho-independent λ T0 and rrnB T1). The genetic modification sequence can cause modification of a sequence of interest in a target genome. In this vector, the genetic modification sequence is comprised in the homology region. The homology region comprises the truncated wcaE coding sequence that replaces the native wcaE sequence in a target genome.

FIG. 1C shows an illustrative vector comprising a positive selection marker gene (e.g., aphA1), a first counter-selection marker (e.g., tetA), a second counter-selection marker (e.g., sacB), a reporter gene (e.g., lacZ for blue/white visual screening with X-Gal), a lactose permease (e.g., lacY for X-Gal transport), a homology region comprising a first homology arm (encoding a region upstream of a sequence of interest coding sequence start codon) and a second homology arm (encoding a region beginning at the sequence of interest coding sequence start codon), and a genetic modification sequence (e.g., a tac promoter and a synthetic RBS). In some embodiments, the vector comprises an OriT (e.g., RP4 OriT), an Ori (e.g., R6K Ori), promoter sequences (e.g., pTetA, BBa_pJ23100, pAphA1, pSacB), RBS sequences, and terminator sequences (e.g., rho-independent λ T0 and rrnB T1). The genetic modification sequence can cause modification of a gene or sequence of interest in a target genome (e.g., insertion of the tac promoter and the synthetic RBS ahead of the qggA coding sequence start codon).

FIG. 2 shows an illustrative graphic depicting a generalized method for modifying and selecting a modified organism using the modification vector. This method comprises i) integration of the vector into the genome of the first population of cells (wild-type) and generating a second population of cells, ii) selecting the second population of cells using a positive selection agent in combination with the positive selection marker encoded within the vector backbone, iii) excision of the vector backbone from the genome of the second population of cells, generating a third population of cells, iv) selecting the third population of cells using the two counter-selection agents in combination with the counter-selection markers encoded within the vector backbone. Generation of the second and third population of cells can each be confirmed through use of a screening agent, in combination with the reporter gene encoded on the vector backbone. The third population of cells will contain a mixture of wild-type and mutant genotypes, dependent upon the site of homologous recombination used to generate the third population of cells.

### DETAILED DESCRIPTION

Many gene disruption strategies may have a propensity to create polar effects in the downstream regions of an inactivated gene. Many selection or counter-selection strategies show variable success across different organisms with diverse genetic backgrounds, owing to varying microbial sensitivity to the relevant selective agents. The variable, strain-specific success of such counter-selection strategies may necessitate screening putative modified strains following counter-selection to ensure the absence of unintended mutations in the strain background.

Provided herein, in some embodiments, are compositions, systems, and methods for producing and selecting a modified organism. The compositions, systems, and methods provided herein can achieve scarless and stable genetic edits in organisms with diverse genetic backgrounds, for example, by coupling a visual reporter gene-mediated screening with counterselection via simultaneous use of two counter-selectable marker genes. The methods provided herein can be alternatives to standard processes for genetic modification of organisms, allowing for more rapid, less costly, more efficient mutant strain construction compared to other tools and methods for engineering organisms. Simultaneous use of two counter-selection markers may reduce the frequency at which a modified organism can escape sensitivity to counter-selective pressure, stemming from varying inherent sensitivity to a counter-selection agent. Additionally, coupled genetic engineering strategies (e.g., selection- or counter-selection-based marker-less deletion systems coupled with systems for rapid verification of correct mutant strain generation) may reduce production times related to engineering bacterial strains.

### Vector and System for Genetic Modification

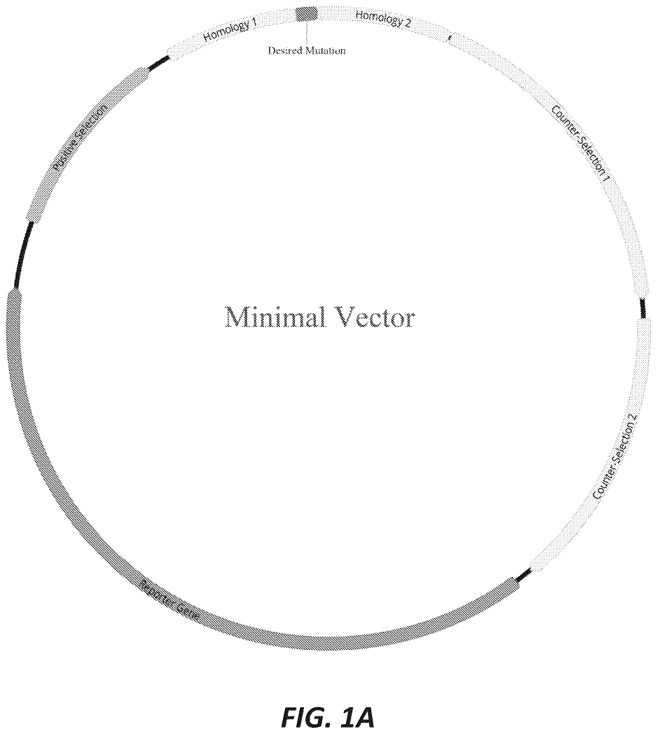

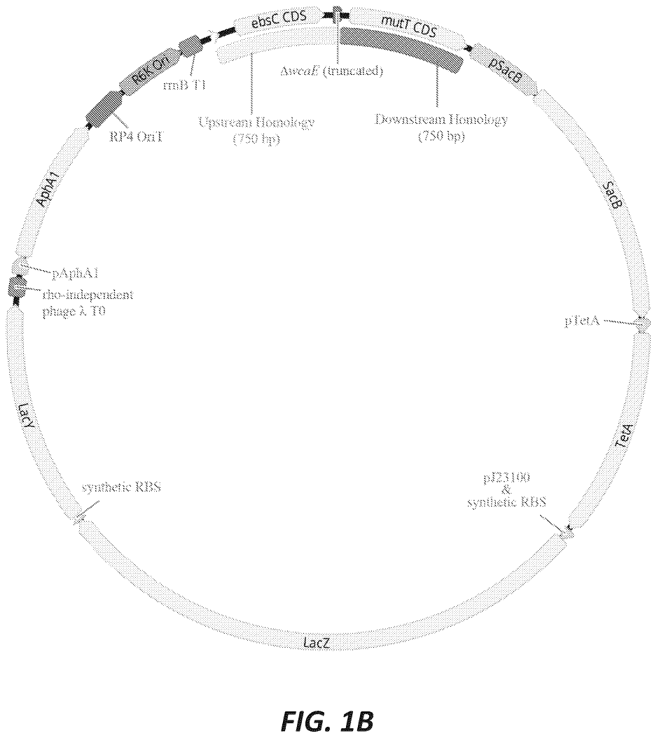

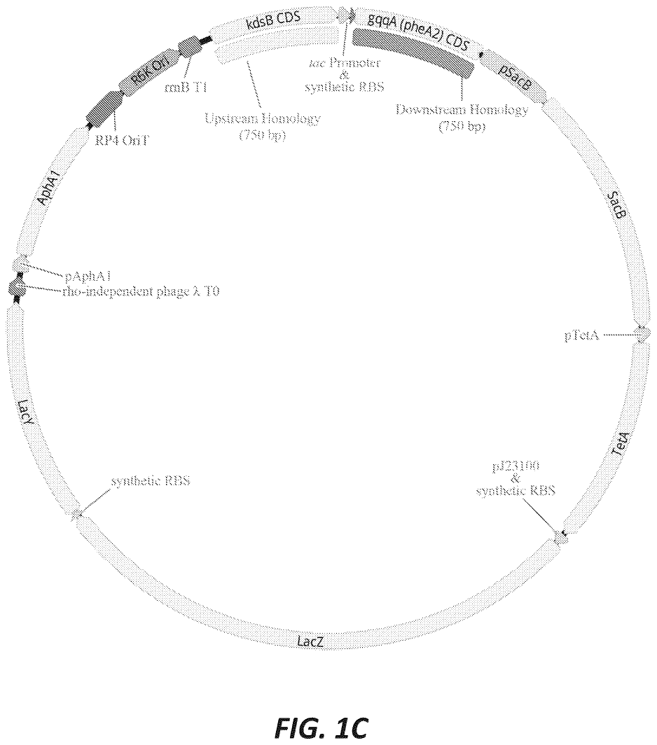

Provided herein is a vector and a system comprising the vector for genetic modification of an organism (FIGS. 1A-C).

In some instances, the vector may comprise a first nucleic acid sequence comprising a positive selection marker gene, a second nucleic acid sequence comprising a first counter-selection marker gene, a third nucleic acid sequence comprising a second counter-selection marker gene, and a fourth nucleic acid sequence comprising a reporter gene. The vector may comprise a fifth nucleic acid sequence comprising a first homology arm that comprises homology with a first (e.g., upstream) portion of a target genomic sequence of an organism targeted for modification. The vector may comprise a sixth nucleic acid sequence comprising a second homology arm that comprises homology with a second (e.g., downstream) portion of said target genomic sequence of said organism targeted for modification. The vector may comprise a genetic modification sequence flanked by said first homology sequence and said second homology sequence.

In some instances, elements of the vector are operably linked to each other. In some instances, elements of the vector are operably linked to a regulatory sequence (e.g., the promoter and the RBS controlling expression of the beta-lactamase reporter gene (lacZ)).

### Positive Selection Marker Genes and Positive Selection Agents

The vector may comprise at least one positive selection marker gene. The positive selection marker may enable cell survival in the presence of a positive selection agent (e.g., positive selection condition). The positive selection agent may be an antibiotic. The positive selection agent may be an environment without a cell survival molecule. The cell survival molecule may be essential to cell survival. The positive selection marker gene may (i) confer resistance to an antibiotic or (ii) create a cell survival molecule. In some instances, the vector comprises a second positive selection marker gene. The second positive selection marker gene may (i) confer resistance to an antibiotic or (ii) create a cell survival molecule.

In some instances, a vector comprising two positive selection marker genes improves activity of the vector in an organism, even when the organism exhibits natural resistance to one of the two positive selection agents that correspond to the two positive selection marker genes. The second positive selection marker gene may improve genetic modification (e.g., operation of the vector) in an organism when the organism exhibits natural resistance to the antibiotic or the organism natively expresses the cell survival molecule. The use of two or more positive selection markers may allow for selection of clones using both positive selection methods simultaneously, thus reducing the possibility that a spontaneous mutation allows growth of the organism in one of the positive selection conditions without genomic incorporation of the vector. Thus, the second positive selection marker may facilitate more efficient operation of the vector. The combination of two positive selection markers may widen the relevant host range of the vector, even when an organism is naturally resistant to one of the two positive selection agents. Thus, the second positive selection marker may facilitate use of the vector in a broad range of organisms.

In some instances, the positive selection marker gene enables antibiotic resistance m an organism with a genetic background lacking a native version of the positive selection marker gene. The antibiotic may be kanamycin, tetracycline, chloramphenicol, ampicillin, carbenicillin, gentamicin, or erythromycin. The positive selection marker gene may encode aminoglycoside phosphotransferase (AphAI) (SEQ ID NO: 1 (Table 1A)), a class A tetracycline resistance protein (TetA) (SEQ ID NO: 2 (Table 1A)), chloramphenicol acetyltransferase, or beta-lactamase. When the positive selection marker gene confers resistance to an antibiotic, the corresponding antibiotic may be used as a positive selection agent. In some instances, use of both positive selection markers aphA1 and tetA allows for more flexibility for positive selection or counter-selection where either kanamycin, tetracycline, or both can be used for selection.

TABLE 1A
Illustrative Genes and Encoded Proteins Relevant to Positive Selection

| SEQ ID | Gene | |
|---|---|---|
| NO: | Identifier | Amino Acid Sequence |
| 1 | aphA1 | MSHIQRETSCSRPRLNSNMDADLYGYKWARDNVGQSGATIYR |
| | | LYGKPDAPELFLKHGKGSVANDVTDEMVRLNWLTEFMPLPTI |
| | | KHFIRTPDDAWLLTTAIPGKTAFQVLEEYPDSGENIVDALAVFL |
| | | RRLHSIPVCNCPFNSDRVFRLAQAQSRMNNGLVDASDFDDERN |
| | | GWPVEQVWKEMHKLLPFSPDSVVTHGDFSLDNLIFDEGKLIGC |
| | | IDVGRVGIADRYQDLAILWNCLGEFSPSLQKRLFQKYGIDNPD |
| | | MNKLQFHLMLDEFF* |
| 2 | tetA | MKSNNALIVILGTVTLDAVGIGLVMPVLPGLLRDIVHSDSIASH |
| | | YGVLLALYALMQFLCAPVLGALSDRFGRRPVLLASLLGATIDY |
| | | AIMATTPVLWILYAGRIVAGITGATGAVAGAYIADITDGEDRA |
| | | RHFGLMSACFGVGMVAGPVAGGLLGAISLHAPFLAAAVLNGL |
| | | NLLLGCFLMQESIIKGERRPMPLRAFNPVSSFRWARGMTIVAAL |
| | | MTVFFIMQLVGQVPAALWVIFGEDRFRWSATMIGLSLAVFGIL |
| | | HALAQAFVTGPATKRFGEKQAIIAGMAADALGYVLLAFATRG |
| | | WMAFPIMILLASGGIGMPALQAMLSRQVDDDHQGQLQGSLAA |
| | | LISLTSITGPLIVTAIYAASASTWNGLAWIVGAALYLVCLPALRR |
| | | GAWSRATST* |

In some instances, the positive selection marker gene encodes a sequence with at least 70%, 75%, 80%, 85%, 90%, 95%, or 100% sequence identity to SEQ ID NO: 1. In some instances, the positive selection marker gene encodes a sequence with at least 90% sequence identity to SEQ ID NO: 1. In some instances, the positive selection marker gene encodes a sequence with at least 70%, 75%, 80%, 85%, 90%, 95%, or 100% sequence identity to SEQ ID NO: 2. In some instances, the positive selection marker gene encodes a sequence with at least 90% sequence identity to SEQ ID NO: 2.

In some instances, the positive selection marker gene enables cell survival in a cell with a genetic background lacking a native version of the positive selection marker gene. The cell survival molecule may be essential to cell survival. The cell survival molecule may support survival of an organism that does not naturally produce the cell survival molecule. The cell survival molecule may be involved in the metabolism of amino acids, carbohydrates, lipids, nucleotides, or vitamins. The cell survival molecule may be orotate phosphoribosyl-transferase, galactokinase 1, thymidylate synthase, orotidine-5′-monophosphate decarboxylase, or uracil phosphoribosyltransferase.

The positive selection marker gene may be involved in the metabolism of amino acids, carbohydrates, lipids, nucleotides, or vitamins. The positive selection marker gene may encode orotate phosphoribosyl-transferase (pyrE) galactokinase 1 (galK), thymidylate synthase (thyA), orotidine-5′-monophosphate decarboxylase (pyrF), or uracil phosphoribosyltransferase (upp). pyrE confers uracil prototrophy onto an organism that lacks a native pyrE (uracil auxotroph). galK enables an organism that lacks a native galK to process galactose when grown in media comprising galactose as the sole carbon source. thyA confers thiamine prototrophy onto an organism that lacks a native thyA. pyrF confers uracil prototrophy onto an organism that lacks a native pyrF (uracil auxotroph).upp confers uracil prototrophy onto an organism that lacks a native upp (uracil auxotroph).

### Counter-Selection Marker Genes and Counter-Selection Conditions

The vector may comprise a first counter-selection marker gene and a second counter-selection marker gene. In some instances, the first counter-selection marker gene inhibits or eliminates growth of the organism when a first counter-selection condition is present. In some instances, the second counter-selection marker gene inhibits or eliminates growth of the organism when a second counter-selection condition is present.

The first counter-selection marker gene may encode levansucrase (SacB) (SEQ ID NO: 3 (Table 1B)), a class A tetracycline resistance protein (TetA) (SEQ ID NO: 2 (Table 1B)), uracil phosphori bosyl-transferase, orotate phosphori bosyl-transferase, cytosine deaminase, or galactokinase 1. The second counter-selection marker gene may encode levansucrase (SacB), a class A tetracycline resistance protein (TetA), uracil phosphoribosyl-transferase, orotate phosphoribosyl-transferase, cytosine deaminase, or galactokinase 1. In some instances, tetA confers fusaric acid sensitivity onto an organism that lacks a native tetA. In some instances, Uracil phosphoribosyl-transferase confers 5-fluorouracil (SFU) confers sensitivity onto an organism that lacks a native uracil phosphoribosyl-transferase. In some instances, Orotate phosphoribosyl-transferase confers 5-fluoroorotic acid onto an organism that lacks native orotate phosphoribosyl-transferase. In some instances, Cytosine deaminase confers 5-fluorocytosine sensitivity onto an organism that lacks native cytosine deaminase. In some instances, Galactokinase 1 confers 2-deoxy-galactose sensitivity onto an organism that lacks native galactokinase 1 by phosphorylating 2-deoxy-galactose to 2-deoxy-galactose-1-phosphate, which is a toxic metabolite. The use of a positive selection marker in addition to the counter-selection markers may allow for more flexibility for positive selection or counter-selection, where either kanamycin, tetracycline, or both can be used for positive selection, and additionally either sucrose, fusaric acid, or both can be used for counter-selection.

TABLE 1B
Illustrative Genes and Encoded Proteins Relevant to Counter Selection

| SEQ ID | Gene | |
|---|---|---|
| NO: | Identifier | Amino Acid Sequence |
| 2 | tetA | MKSNNALIVILGTVTLDAVGIGLVMPVLPGLLRDIVHSDSIAS |
| | | HYGVLLALYALMQFLCAPVLGALSDRFGRRPVLLASLLGATI |
| | | DYAIMATTPVLWILYAGRIVAGITGATGAVAGAYIADITDGE |
| | | DRARHFGLMSACFGVGMVAGPVAGGLLGAISLHAPFLAAAV |
| | | LNGLNLLLGCFLMQESHKGERRPMPLRAFNPVSSFRWARGM |
| | | TIVAALMTVFFIMQLVGQVPAALWVIFGEDRFRWSATMIGLS |
| | | LAVFGILHALAQAFVTGPATKRFGEKQAIIAGMAADALGYVL |
| | | LAFATRGWMAFPIMILLASGGIGMPALQAMLSRQVDDDHQG |
| | | QLQGSLAALISLTSITGPLIVTAIYAASASTWNGLAWIVGAAL |
| | | YLVCLPALRRGAWSRATST* |
| 3 | sacB | MNIKKFAKQATVLTFTTALLAGGATQAFAKETNQKPYKETY |
| | | GISHITRHDMLQIPEQQKNEKYQVPEFDSSTIKNISSAKGLDV |
| | | WDSWPLQNADGTVANYHGYHIVFALAGDPKNADDTSIYMF |
| | | YQKVGETSIDSWKNAGRVFKDSDKFDANDSILKDQTQEWSG |
| | | SATFTSDGKIRLFYTDFSGKHYGKQTLTTAQVNVSASDSSLNI |
| | | NGVEDYKSIFDGDGKTYQNVQQFIDEGNYSSGDNHTLRDPHY |
| | | VEDKGHKYLVFEANTGTEDGYQGEESLFNKAYYGKSTSFFR |
| | | QESQKLLQSDKKRTAELANGALGMIELNDDYTLKKVMKPLI |
| | | ASNTVTDEIERANVFKMNGKWYLFTDSRGSKMTIDGITSNDI |
| | | YMLGYVSNSLTGPYKPLNKTGLVLKMDLDPNDVTFTYSHFA |
| | | VPQAKGNNVVITSYMTNRGFYADKQSTFAPSFLLNIKGKKTS |
| | | VVKDSILEQGQLTVNK* |

In some instances, the first counter-selection marker gene or the second counter-selection marker gene encodes a sequence with at least 70%, 75%, 80%, 85%, 90%, 95%, or 100% sequence identity to SEQ ID NO: 2. In some instances, the first counter-selection marker gene or the second counter-selection marker gene encodes a sequence with at least 90% sequence identity to SEQ ID NO: 2. In some instances, the first counter-selection marker gene or the second counter-selection marker gene encodes a sequence with at least 70%, 75%, 80%, 85%, 90%, 95%, or 100% sequence identity to SEQ ID NO: 3. In some instances, the first counter-selection marker gene or the second counter-selection marker gene encodes a sequence with at least 90% sequence identity to SEQ ID NO: 3.

In some instances, the presence of the first counter-selection condition reduces the possibility that a mutation in either the first counter-selection marker gene or the second counter-selection marker gene allows for growth of the organism without excision of the vector backbone. In some instances, the presence of the second counter-selection condition reduces the possibility that a mutation in either the first counter-selection marker gene or the second counter-selection marker gene allows for growth of the organism without excision of the vector backbone. The mutation may be a frameshift mutation, a missense mutation, or a nonsense mutation.

In some instances, the presence of the first counter-selection condition reduces the possibility that a mutation in either the first counter-selection marker gene or the second counter-selection marker gene allows for growth of the organism without excision of the backbone of the vector compared to a vector that does not comprise both the first counter-selection marker gene and the second counter-selection marker gene. The presence of the first counter-selection condition may reduce the possibility that a mutation in either the first counter-selection marker gene or the second counter-selection marker gene allows for growth of the organism without excision of the backbone of the vector by at least 5%, 10%, 15%, 20%, 25%, 30%, 35%, 40%, 45%, 50%, 55%, 60%, 65%, 70%, 75%, 80%, 85%, 90%, or 95% as compared to a vector that does not comprise both the first counter-selection marker gene and the second counter-selection marker gene.

In some instances, the presence of the second counter-selection condition reduces the possibility that a mutation in either the first counter-selection marker gene or the second counter-selection marker gene allows for growth of the organism without excision of the backbone of the vector compared to a vector that does not comprise both the first counter-selection marker gene and the second counter-selection marker gene. The presence of the second counter-selection condition may reduce the possibility that a mutation in either the first counter-selection marker gene or the second counter-selection marker gene allows for growth of the organism without excision of the backbone of the vector by at least 5%, 10%, 15%, 20%, 25%, 30%, 35%, 40%, 45%, 50%, 55%, 60%, 65%, 70%, 75%, 80%, 85%, 90%, or 95% as compared to a vector that does not comprise both the first counter-selection marker gene and the second counter-selection marker gene.

In some instances, the presence of the first counter-selection condition and the presence of the second counter-selection condition reduces the possibility that a mutation in either the first counter-selection marker gene or the second counter-selection marker gene allows for growth of the organism without excision of the backbone. The mutation may be a frameshift mutation, a missense mutation, or a nonsense mutation. The presence of the first counter-selection condition may reduce the possibility that a mutation in either the first counter-selection marker gene or the second counter-selection marker gene allows for growth of the organism without excision of the backbone of the vector by at least 5%, 10%, 15%, 20%, 25%, 30%, 35%, 40%, 45%, 50%, 55%, 60%, 65%, 70%, 75%, 80%, 85%, 90%, or 95% as compared to a vector that does not comprise both the first counter-selection marker gene and the second counter-selection marker gene.

In some instances, presence of the first counter-selection condition or the second counter-selection condition may reduce the possibility that a spontaneous mutation in either the first counter-selection marker gene or the second counter-selection marker gene allows for growth of the organism without excision of the backbone of the vector. The spontaneous mutation may be predicted via a spontaneous mutation rate (e.g., frequency that a spontaneous mutation disrupts function of a gene). If the frequency that a spontaneous mutation disrupts function of the first counter-selection gene is 10⁻³ and the frequency that a spontaneous mutation disrupts function of the second counter-selection gene is 10⁻³, then the possibility that a mutation causing loss of function in either the first counter-selection marker gene or the second counter-selection marker gene allows for growth of the organism without excision of the backbone of the vector in the presence of the first counter selection condition and the second counter selection condition may be 10⁻³×10⁻³.

The combination of two counter-selection markers may be more sensitive than and may reduce the frequency of false-positives more than the use of either counter-selection strategy alone. The combination of two counter-selection markers may reduce the possibility that a frameshift, missense, or nonsense mutation in the counter-selectable marker sequence allows for growth on the counter-selection condition without excision of the vector backbone.

### Reporter Genes and Visual Phenotypes

The vector may comprise a reporter gene. In some instances, the reporter gene may comprise a visual reporter gene. In some instances, the reporter gene may generate a visual phenotype. The reporter gene may comprise lacZ (SEQ ID NO: 4 (Table 1C)), a lacZ homolog, a gene encoding green fluorescent protein (GFP), a gene encoding red fluorescent protein (RFP), or a gene encoding luciferase (luc). lacZ encodes the β-galactosidase protein.

TABLE 1C
Illustrative Genes and Encoded Proteins Relevant to Visual Phenotype

| SEQ ID | Gene | |
|---|---|---|
| NO: | Identifier | Amino Acid Sequence |
| 4 | lacZ | MTMITDSLAVVLQRRDWENPGVTQLNRLAAHPPFASWRNSEE |
| | | ARTDRPSQQLRSLNGEWRFAWFPAPEAVPESWLECDLPEADTV |
| | | VVPSNWQMHGYDAPIYTNVTYPITVNPPFVPTENPTGCYSLTF |
| | | NVDESWLQEGQTRIIFDGVNSAFHLWCNGRWVGYGQDSRLPS |
| | | EFDLSAFLRAGENRLAVMVLRWSDGSYLEDQDMWRMSGIFRD |
| | | VSLLHKPTTQISDFHVATRENDDFSRAVLEAEVQMCGELRDYL |
| | | RVTVSLWQGETQVASGTAPFGGEIIDERGGYADRVTLRLNVEN |
| | | PKLWSAEIPNLYRAVVELHTADGTLIEAEACDVGFREVRIENGL |
| | | LLLNGKPLLIRGVNRHEHHPLHGQVMDEQTMVQDILLMKQNN |
| | | FNAVRCSHYPNHPLWYTLCDRYGLYVVDEANIETHGMVPMN |
| | | RLTDDPRWLPAMSERVTRMVQRDRNHPSVIIWSLGNESGHGA |
| | | NHDALYRWIKSVDPSRPVQYEGGGADTTATDIICPMYARVDED |
| | | QPFPAVPKWSIKKWLSLPGETRPLILCEYAHAMGNSLGGFAKY |
| | | WQAFRQYPRLQGGFVWDWVDQSLIKYDENGNPWSAYGGDFG |
| | | DTPNDRQFCMNGLVFADRTPHPALTEAKHQQQFFQFRLSGQTI |
| | | EVTSEYLFRHSDNELLHWMVALDGKPLASGEVPLDVAPQGKQ |
| | | LIELPELPQPESAGQLWLTVRVVQPNATAWSEAGHISAWQQW |
| | | RLAENLSVTLPAASHAIPHLTTSEMDFCIELGNKRWQFNRQSGF |
| | | LSQMWIGDKKQLLTPLRDQFTRAPLDNDIGVSEATRIDPNAWV |
| | | ERWKAAGHYQAEAALLQCTADTLADAVLITTAHAWQHQGKT |
| | | LFISRKTYRIDGSGQMAITVDVEVASDTPHPARIGLNCQLAQVA |
| | | ERVNWLGLGPQENYPDRLTAACFDRWDLPLSDMYTPYVFPSE |
| | | NGLRCGTRELNYGPHQWRGDFQFNISRYSQQQLMETSHRHLL |
| | | HAEEGTWLNIDGFHMGIGGDDSWSPSVSAEFQLSAGRYHYQL |
| | | VWCQK* |

In some instances, the reporter gene encodes a sequence with at least 70%, 75%, 80%, 85%, 90%, 95%, or 100% sequence identity to SEQ ID NO: 4. In some instances, the reporter gene encodes a sequence with at least 90% sequence identity to SEQ ID NO: 4.

The visual phenotype may present when a visual screening agent is present. Presence of the visual phenotype may indicate the vector successfully integrated into an organism. Absence of the visual phenotype may indicate either (i) the vector did not successfully integrate into an organism (e.g., a wild-type organism) or (ii) the vector backbone successfully excised from an organism after the vector successfully integrated into the organism (e.g., a successfully modified organism). Thus, presence or absence of the visual phenotype may indicate both integration of the vector into a target genome and excision of the vector backbone from the target genome.

When the reporter gene is lacZ and the visual screening agent is 5-bromo-4-chloro-3-indolyl-β-D-galactopyranoside (X-Gal), the visual phenotype may be blue or white colonies. lacZ-mediated blue/white colony screening may allow for immediate assessment of integration of the vector into the target (e.g., wild-type) genome following positive selection. lacZ-mediated blue/white colony screening may allow for immediate assessment of excision of the vector backbone from the target genome following counter-selection. Immediate visual assessment may provide cost and time savings for making genomic edits in an organism by eliminating assessing vector integration via molecular methods (e.g., PCR) following positive selection. Immediate visual assessment may provide cost and time savings for making genomic edits in an organism by eliminating screening against loss of positive selection resistance or assessing vector backbone excision via molecular methods (e.g., PCR) following counter-selection.

### Homology Arms

The vector may comprise a first homology arm that comprises homology with a first portion of a target genomic sequence of an organism targeted for modification (e.g., the first homology arm shares homology with a first (e.g., upstream or 5′) region of the target genomic sequence). The vector may comprise a second homology arm that comprises homology with a second portion of a target genomic sequence of an organism targeted for modification (e.g., the second homology arm shares homology with a second (e.g., downstream or 3′) region of the target genomic sequence). The first homology arm and the second homology arm (e.g., the homology arms) may comprise homology with the target genomic sequence of the organism. The target genomic sequence may be comprised in a target genome targeted for modification. The homology arms may each be at least 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, or more, in length.

The homology arms may enable vector integration into and vector backbone excision from the target genome. The homology arms may allow integration of the vector into the target genomic sequence. The homology arms may allow excision of a backbone of the vector from the target genomic sequence after the integration. The vector backbone may comprise the vector sequences excluding the genetic modification sequence and the homology region. Once the homology arms facilitate integration of the vector into the target genomic sequence, the homology arms may then facilitate excision of the backbone of the vector from the target genomic sequence.

When the integration occurs in the presence of a positive selection agent, the organism may continue growing in the presence of the positive selection agent. When the excision occurs in the presence of a counter-selection condition, the organism may continue growing in the presence of the counter-selection condition. When the integration occurs in the presence of the visual screening agent, the organism may exhibit the visual phenotype. When the excision occurs in the presence of the visual screening agent, the organism may not exhibit the visual phenotype.

### Gene Modification Sequence

The vector may comprise a genetic modification sequence. The genetic modification sequence may comprise a sequence of interest. The sequence of interest may comprise a gene of interest. The genetic modification sequence may comprise another vector element (e.g., a promoter, a ribosomal binding site (RBS), etc.). The genetic modification sequence may be empty. In some instances, the genetic modification sequence is flanked by the first homology sequence and the second homology sequence. The vector backbone may not comprise the genetic modification sequence. The genetic modification sequence may cause modification of a sequence of interest in a target genome. The genetic modification sequence may cause mutation, knockout, truncation, knockdown, disruption, mitigation, inhibition, deletion, insertion, enhancement, or substitution of a sequence of interest in a target genome. The genetic modification sequence may cause insertion, deletion, or substitution of a single nucleotide in a target genome; insertion, deletion, or substitution of two or more nucleotides in a target genome; or two or more non-contiguous insertions, deletions, or substitutions in a target genome.

The genetic modification sequence may cause modification of a sequence of interest in a target genome via homologous recombination mediated by the homology arms. When the genetic modification causes a deletion of a sequence of interest from the target genome, the homology arms may be fused flanking DNA segments encoded immediately upstream and downstream of the sequence of interest (FIG. 1B). The genetic modification sequence may be empty. The vector may cause deletion of a sequence of interest between the two homology arms as the sequences of the two homology arms exist in the target genome.

When the genetic modification causes an insertion of a sequence of interest into the target genome, the genetic modification sequence comprising the sequence of interest may be between the homology arms (FIG. 1C). The genetic modification sequence may comprise a nucleotide sequence. The vector may cause insertion of the genetic modification sequence between the two homology arms as the sequences of the two homology arms exist in the target genome.

The genetic modification sequence may cause modification of another vector element (e.g., a promoter, a ribosomal binding site (RBS), etc.) in a target genome via homologous recombination mediated by the homology arms. The genetic modification sequence may cause an insertion of another vector element (e.g., a promoter, a ribosomal binding site (RBS), etc.) in a target genome. When the genetic modification sequence causes insertion of a promoter or an RBS, the insertion may be upstream of a sequence of interest targeted for upregulation or constitutive expression. When the genetic modification sequence causes insertion of a terminator, the insertion may be downstream of a gene or sequence of interest.

### Other Vector Elements

The vector may comprise a promoter, a ribosomal binding site (RBS), a transcription terminator, an origin of transference (OriT), an origin of replication (Ori), a multiple cloning site (MCS), or a transporter. The elements of the vector may be operably linked.

In some instances, the promoter comprises a native promoter, a hybrid promoter, or a synthetic promoter designed in silico. The native promoter may comprise a native promoter for sacB (SEQ ID NO: 5 (Table 1D)), a native promoter for tetA (SEQ ID NO: 6 (Table 1D)), or a native promoter for aphA1 (SEQ ID NO: 7 (Table 1D)). The hybrid promoter may comprise a tac promoter (SEQ ID NO: 8 (Table 1D)). The synthetic promoter may comprise a promoter from a promoter collection. The synthetic promoter may comprise a promoter from the Anderson promoter collection (e.g., a BBa_J23100 promoter (SEQ ID NO: 9 (Table 1D))).

TABLE 1D
Illustrative Promoters

| SEQ ID | Gene | |
|---|---|---|
| NO: | Identifier | Nucleotide Sequence |
| 5 | sacB | CACATATACCTGCCGTTCACTATTATTTAGTGAAATGAGATATTAT |
| | promoter | GATATTTTCTGAATTGTGATTAAAAAGGCAACTTTATGCCCATG |
| | | CAACAGAAACTATAAAAAATACAGAGAATGAAAAGAAACAGATA |
| | | GATTTTTTAGTTCTTTAGGCCCGTAGTCTGCAAATCCTTTTATG |
| | | ATTTTCTATCAAACAAAAGAGGAAAATAGACCAGTTGCAATCCAA |
| | | ACGAGAGTCTAATAGAATGAGGTCGAAAAGTAAATCGCGCGGGT |
| | | TTGTTACTGATAAAGCAGGCAAGACCTAAAATGTGTAAAGGGCA |
| | | AAGTGTATACTTTGGCGTCACCCCTTACATATTTTAGGTCTTTTT |
| | | TTATGTGCGTAACTAACTTGCCATCTTCAAACAGGAGGGCTGG |
| | | AAGAAGCAGACCGCTAACACAGTACATAAAAAAGGAGACATGAA |
| | | CG |
| 6 | tetA | TTGACAGCTTATCATCGATAAACTGTAATGCGGTAGTTTATCACAG |
| | promoter | TTAAATTGCTAACGCAGTCAGGCACCGTGT |
| 7 | aphA1 | TTGTGTCTCAAAATCTCTGATGTTACATTGCACAAGATAAAAATAT |
| | promoter | ATCATCATGAACAATAAAACTGTCTGCTTACATAAACAGTAATA |
| | | CAAGGGGTGTT |
| 8 | tac | GAGCTGTTGACAATTAATCATCGGCTCGTATAATGTGTGGAATTGT |
| | promoter | GAGCGGATAACAATTTCACAC |
| 9 | BBa_J23100 | TTGACGGCTAGCTCAGTCCTAGGTACAGTGCTAGCCAAGAAGCITT |
| | promoter | ATTTTGGGGAGACACATC |

In some instances, the promoter encodes a sequence with at least 70%, 75%, 80%, 85%, 90%, 95%, or 100% sequence identity to SEQ ID NO: 5. In some instances, the promoter encodes a sequence with at least 90% sequence identity to SEQ ID NO: 5. In some instances, the promoter encodes a sequence with at least 70%, 75%, 80%, 85%, 90%, 95%, or 100% sequence identity to SEQ ID NO: 6. In some instances, the promoter encodes a sequence with at least 90% sequence identity to SEQ ID NO: 6. In some instances, the promoter encodes a sequence with at least 70%, 75%, 80%, 85%, 90%, 95%, or 100% sequence identity to SEQ ID NO: 7 In some instances, the promoter encodes a sequence with at least 90% sequence identity to SEQ ID NO: 7. In some instances, the promoter encodes a sequence with at least 70%, 75%, 80%, 85%, 90%, 95%, or 100% sequence identity to SEQ ID NO: 8. In some instances, the promoter encodes a sequence with at least 90% sequence identity to SEQ ID NO: 8. In some instances, the promoter encodes a sequence with at least 70%, 75%, 80%, 85%, 90%, 95%, or 100% sequence identity to SEQ ID NO: 9. In some instances, the promoter encodes a sequence with at least 90% sequence identity to SEQ ID NO: 9.

In some instances, the RBS comprises a nucleotide sequence derived from a Gram-negative bacteria or a synthetic RBS designed in silico. The RBS may be specific to the vector. The RBS may be specific to a sequence of interest. The RBS may be specific to constitutive expression of a sequence of interest. The RBS may comprise at least one motif that targets ribosome initiation at the RBS.

In some instances, the transcription terminator comprises an rrnB terminator (SEQ ID NO: 10 (Table IE)), a rho-independent terminator of phage) . . . (SEQ ID NO: 11 (Table IE)), a nucleotide sequence derived from AAB, a nucleotide sequence derived from a Gram-negative bacteria, or a transcriptional terminator sequence designed in silico.

TABLE 1E
Illustrative Terminators

| SEQ ID | Gene | |
|---|---|---|
| NO: | Identifier | Nucleotide Sequence |
| 10 | rrnB | CAGCTGTCTAGGGCGGCGGATTTGTCCTACTCAGGAGAGCGTTCACCG |
| | terminator | ACAAACAACAGATAAAACGAAAGGCCCAGTCTTTCGACTGAGCCTTTCGT |
| | | TTTATTTGATGCCT |
| 11 | rho- | CTTGGACTCCTGTTGATAGATCCAGTAATGACCTCAGAACTCCATCTGGA |
| | independent | TTTGTTCAGAACGCTCGGTTGCCGCCGGGCGTTTTTTATTGGTGAGAA |
| | phage λ | TCCAG |
| | terminator | |

In some instances, the transcription terminator encodes a sequence with at least 70%, 75%, 80%, 85%, 90%, 95%, or 100% sequence identity to SEQ ID NO: 10. In some instances, the transcription terminator encodes a sequence with at least 90% sequence identity to SEQ ID NO: 10. In some instances, the transcription terminator encodes a sequence with at least 70%, 75%, 80%, 85%, 90%, 95%, or 100% sequence identity to SEQ ID NO: 11. In some instances, the transcription terminator encodes a sequence with at least 90% sequence identity to SEQ ID NO: 11.

In some instances, the OriT enables conjugative transfer of nucleic acids between bacterial cells. The OriT may be derived from an RP4 plasmid (SEQ ID NO: 12 (Table IF)).

TABLE 1F
Illustrative OriTs

| SEQ ID | Gene | |
|---|---|---|
| NO: | Identifier | Nucleotide Sequence |
| 12 | RP4 OriT | CTTTTCCGCTGCATAACCCTGCTTCGGGGTCATTATAGCGAT |
| | | TTTTTCGGTATATCCATCCTTTTTCGCACGATATACAGGATTT |
| | | TGCCAAAGGGTTCGTGTAGACTTTCCTTGGTGTATCCAACGG |
| | | CGTCAGCCGGGCAGGATAGGTGAAGTAGGCCCACCCGCGAG |
| | | CGGGTGTTCCTTCTTCACTGTCCCTTATTCGCACCTGGCGGT |
| | | GCTCAACGGGAATCCTGCTCTGCGAGGCTGGCCGTA |

In some instances, the OriT encodes a sequence with at least 70%, 75%, 80%, 85%, 90%, 95%, or 100% sequence identity to SEQ ID NO: 12. In some instances, the OriT encodes a sequence with at least 90% sequence identity to SEQ ID NO: 12.

In some instances, the Ori enables vector replication in a microorganism used to store, modify, or produce said vector. The Oni may be unable to facilitate replication of the vector in a genetic background subject to genomic modification. The Oni may be unable to facilitate replication of the vector in the organism. The Oni may be R6Kγ (SEQ ID NO: 13 (Table IG)).

TABLE 1G
Illustrative Oris

| SEQ ID | Gene | |
|---|---|---|
| NO: | Identifier | Nucleotide Sequence |
| 13 | R6Kγ Ori | GATCTGAAGATCAGCAGTTCAACCTGTTGATAGTACGTACTA |
| | | AGCTCTCATGTTTCACGTACTAAGCTCTCATGTTTAACGTAC |
| | | TAAGCTCTCATGTTTAACGAACTAAACCCTCATGGCTAACGT |
| | | ACTAAGCTCTCATGGCTAACGTACTAAGCTCTCATGTTTCAC |
| | | GTACTAAGCTCTCATGTTTGAACAATAAAATTAATATAAATC |
| | | AGCAACTTAAATAGCCTCTAAGGTTTTAAGTTTTATAAGAAA |
| | | AAAAAGAATATATAAGGCTTTTAAAGCCTTTAAGGTTTAAC |
| | | GGTTGTGGACAACAAGCCAGGGATGTAACGCACTGAGAAGC |
| | | CCTTAGAGCCTCTCAAAGCAATTTTGAGTGACACAGGAACA |
| | | CTTAACGGCTGACATGG |

In some instances, the Ori encodes a sequence with at least 70%, 75%, 80%, 85%, 90%, 95%, or 100% sequence identity to SEQ ID NO: 13. In some instances, the Ori encodes a sequence with at least 90% sequence identity to SEQ ID NO: 13.

In some instances, the MCS encodes at least two restriction enzyme recognition sites. The at least two restriction enzyme recognition sites may comprise restriction enzyme recognition sites for SacI, SmaI, BamHI, HindIII, XbaI, SalI, PstI, or SphI. The at least two restriction enzyme recognition sites may comprise restriction enzyme recognition sites for AclI, HindIII, SspI, MIuCI, PciI, Agel, BfuAI, BspMI, SexAI, MluI, BceAI, HpyCH4IV, HpyCH4III, BaeI, BsaXI, AflIII, Spel, BsrI, BmrI, BglII, Afel, AluI, Stul, Scal, ClaI, BspDI, PI-SceI, NsiI, AseI, Swal, CspCI, MfeI, PaqCI, BssSI-v2, Nb.BssSI, BmgBI, PmlI, DraIII, AleI-v2, EcoPi5I, PvuII, AlwNI, BtsI, MutI, NdeI, FatI, NiaIII, MsII, XcmI, BstXI, PflMI, BccI, NcoI, BseYI, FauI, SmaI, XmaI, TspMI, Nt.CviPII, AciI, SAcII, BsrBI, HpaII, MspI, StyD4I, ScrFI, BsaJI, BslI, BtgI, NciI, AvrII, MnlI, BbvCI, Nb.BbvCI, Nt.BbvCI, SbfI, Bpu10I, Bsu36I, EcoNI, HpyAV, BstNI, PspGI, Styl, BegI, PvuI, BstUI, EagI, RsrII, BsiEI, BsiWI, BsmBI-v2, Esp3I, Hpy99I, MspA1I, MspJI, SgrAI, BfaI, BspCNI, PaeR7I, XhoI, Earl, Acul, PstI, BpmI, DdeI, SfcI, AflII, BpuEI, SmIlI, Aval, BsoBI, MboII, BbsI, XmnI, BsmI, Nb.BsmI, EcoRI, HgaI, AatII, ZraI, PflFI, Tth111I, PshAI, AhdI, DrdI, SacI, Eco53kI, BseRI, PleI, MlyI, Nt.BstNBI, HinfI, EcoRV, Sau3AI, MboI, DpnII, DpnI, BsaBI, Tfil, BsrDI, Nb.BsrDI, BbvI, BtsI-v2, Nb.BtsI, BstAPI, SfaNI, SphI, Srfl, NmeAIII, NaeI, NgoMIV, BgII, AsiSI, BtgZI, HhaI, HinP1I, BssHII, NotI, Fnu4HI, Cac8I, MwoI, NheI, BmtI, BspQI, SapI, Nt.BspQI, BIpI, TseI, ApeKI, Bsp1286I, AlwI, BamHI, Nt.AlwI, FokI, BtsCI, HaeIII, FseI, SfiI, NarI, KasI, PluTI, SfoI, AscI, EciI, BsmFI, PspOMI, Apal, Sau96I, NlaIV, KpnI, Acc65I, BsaI, HphI, BstEII, AvaII, BanI, BaeGI, BsaHI, BanII, CviQI, RsaI, BstZ17I, BciVI, SalI, BsmAI, BcoDI, Nt.BsmAI, ApaLI, BsgI, AccI, Hpy166II, Tsp45I, HpaI, PmeI, HincII, BsiHKAI, TspRI, ApoI, NspI, BsrFI-v2, BstYI, HaeII, CviKI-1, EcoO109I, PpuMI, I-Ceul, SnaBI, I-SceI, BspHI, BspEI, MmeI, TaqI-v2, NruI, Hpy188I, Hpy188III, XbaI, BcII, HpyCH4V, FspI, PI-PspI, MscI, BsrGI, MseI, PacI, PsiI-v2, BstBI, Dral, PspXI, BsaWI, BsaAI, or EaeI.

In some instances, the transporter facilitates cellular membrane transportation of a substrate. Production of a visual phenotype related to the reporter gene may utilize the substrate to produce the visual phenotype. The transporter gene may be lacY (SEQ ID NO: 14 (Table IH)). lacY encodes a lactose permease (e.g., LacY) which may transport X-Gal across the cellular membrane of an organism.

TABLE 1H
Illustrative Transporters

| SEQ ID | Gene | |
|---|---|---|
| NO: | Identifier | Amino Acid Sequence |
| 14 | lacY | MYYLKNTNFWMFGLFFFFYFFIMGAYFPFFPIWLHDINHISKSD |
| | transporter | TGIIFAAISLFSLLFQPLFGLLSDKLGLRKYLLWIITGMLVMFAPF |
| | | FIFIFGPLLQYNILVGSIVGGIYLGFCFNAGAPAVEAFIEKVSRRS |
| | | NFEFGRARMFGCVGWALCASIVGIMFTINNQFVFWLGSGCALI |
| | | LAVLLFFAKTDAPSSATVANAVGANHSAFSLKLALELFRQPKL |
| | | WFLSLYVIGVSCTYDVFDQQFANFFTSFFATGEQGTRVFGYVT |
| | | TMGELLNASIMFFAPLIINRIGGKNALLLAGTIMSVRIIGSSFATS |
| | | ALEVVILKTLHMFEVPFLLVGCFKYITSQFEVRFSATIYLVCFCF |
| | | FKQLAMIFMSVLAGNMYESIGFQGAYLVLGLVALGFTLISVFTL |
| | | SGPGPLSLLRRQVNEVA* |

In some instances, the transporter encodes a sequence with at least 70%, 75%, 80%, 85%, 90%, 95%, or 100% sequence identity to SEQ ID NO: 14. In some instances, the transporter encodes a sequence with at least 90% sequence identity to SEQ ID NO: 14.

### Introduction to an Organism

In some instances, the vector may be introduced to an organism. The vector may be introduced to the organism to modify the organism or produce a genetic modification of the organism. The vector may be introduced to the organism via electroporation, conjugation, transduction, transfection, or other suitable methods.

The organism may be a bacterium. The organism may be a Gram-negative bacterium. The organism may be a Zymomonas bacterium, a Pseudomonas bacterium, a Lactobacillus bacterium, a Escherichia bacterium, or an Acinetobacter bacterium.

In some instances, the organism is within the family Acetobacteraceae (e.g., an acetic acid bacterium (AAB)). Acetic acid bacteria are widespread and versatile organisms that produce numerous natural and industrially valuable products. The organism may be of a species from the genera Acetobacter, Gluconacetobacter, Gluconobacter, or Komagataeibacter. In some instances, the organism is from the genus Komagataeibacter. In some instances, the organism is from the species Komagataeibacter europaeus. In some instances, the organism may be Komagataeibacter europaeus LMG 1521. The organism may be capable of oxidizing ethanol to produce acetic acid.

In some instances, the organism may have a genetic background that enables effective genetic modification by the vector. The organism may lack a native version of a positive selection marker gene. The organism may be sensitive to the positive selection agent. The organism may lack a native version of one or both counter-selection marker genes. The organism may be sensitive to the function of one or both counter-selection marker genes in the presence of one or both counter-selection agents. The organism may lack a genetic background capable of supporting the Ori in replicating the vector in the organism.

### Methods for Selecting a Modified Organism

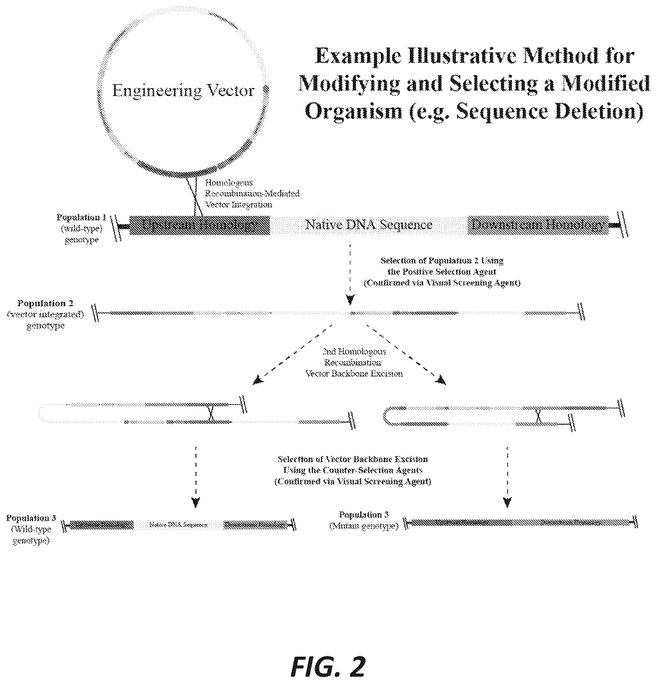

Provided herein is a method for selecting a modified organism. In some instances, a vector may be introduced to an organism, then subjected to positive selection, and then subjected to counter-selection (FIG. 2).

In some instances, the method comprises (a) introducing a vector to a first population of cells. The vector is further described herein. Introducing the vector to the first population may generate a second population of cells. The vector may be integrated into a target genome of the second population. In some instances, the method comprises (b) growing the first population and the second population in a first media comprising a positive selection agent and a visual screening agent. In some instances, the method comprises (c) selecting the second population based on a visual phenotype. In some instances, the method comprises (d) growing the second population in a second media comprising a first counter-selection condition, a second counter-selection condition, and the visual screening agent. Growing the second population in the second media may generate a third population of cells. A backbone of the vector may be excised from a target genome of the third population. The third population may comprise the modified organism. In some instances, the method comprises (e) selecting the third population based on the visual phenotype. In some instances, the method comprises (f) screening the selected third population for a genetic modification sequence.

In some instances, introducing the vector produces at least I %, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or more, fewer false-positives than a comparable method where the vector does not comprise the positive selection marker gene, the first counter-selection marker gene, the second counter-selection marker gene, or the reporter gene.

In some instances, introducing the vector produces the modified organism in fewer steps than introducing a vector not comprising the positive selection marker gene, the first counter-selection marker gene, the second counter-selection marker gene, or the reporter gene. A vector comprising the reporter gene may allow for selection of the second population without intermittent molecular screening (e.g., PCR) of the second population to confirm integration of the vector into the backbone. A vector comprising the reporter gene may allow for selection of the third population without molecular screening or phenotypic screening of the third population to confirm excision of the vector backbone.

In some instances, introducing the vector produces the modified organism in less time than introducing a vector not comprising the positive selection marker gene, the first counter-selection marker gene, the second counter-selection marker gene, or the reporter gene. Introducing the vector may produce the modified organism in less time on the order of hours or days. In some instances, introducing the vector produces the modified organism at a lower cost than introducing a vector not comprising the positive selection marker gene, the first counter-selection marker gene, the second counter-selection marker gene, or the reporter gene.

The systems and methods described herein may be used iteratively to produce and select a modified organism comprising multiple mutations. In some instances, engineering the organism may be performed iteratively. A series of vectors, each comprising the same backbone and each comprising unique homology arms and unique genetic modification sequences, may be used iteratively to create multiple mutations in an organism. A first vector may be used to engineer an organism as described herein, to create a first mutation. A second vector, comprising the same backbone as a first vector but different homology arms and a different genetic modification sequence from the first vector, may then be used to engineer the organism again, such that the organism comprises the first mutation and a second mutation.

### Integration: The First Population and Generating the Second Population

In some instances, the method comprises (a) introducing a vector to a first population of cells. The vector is further described herein. Introducing a vector may be via electroporation, conjugation, transduction, transfection, or other suitable methods. The cell may be a Gram-negative bacterium. The cell may be a Zymomonas bacterium, a Pseudomonas bacterium, a Lactobacillus bacterium, a Escherichia bacterium, or an Acinetobacter bacterium. The cell may be an Acetobacteraceae. The cell may be an Acetobacter, a Gluconacetobacter, a Gluconobacter, or a Komagataeibacter.

In some instances, introducing the vector to the first population generates a second population of cells. The vector may be integrated into a target genome of the second population. Introducing the vector to the first population may generate a genetic modification in at least one cell of the first population, thus generating the second population. Introducing the vector to the first population may allow the vector to integrate into a target genome of the first population, thus generating the second population. Integration of the vector into the target genome of the second population may introduce the genetic modification sequence to the target genome of the second population.

In some instances, integration of the vector into the target genome of the second population is via homologous recombination. The homology arms may facilitate integration of the vector into the target genomic sequence and excision of the vector backbone from the target genomic sequence via homologous recombination. The homologous recombination may occur via endogenous homologous recombination machinery (e.g., native RecA) or via heterologous homologous recombination machinery (e.g., a non-native RecA ortholog).

The integration may be via endogenous homologous recombination machinery. The endogenous machinery may be natively expressed by the organism. Expression or activity of the endogenous homologous recombination machinery may be enhanced. Expression or activity of the endogenous homologous recombination machinery may be enhanced by a genetic modification sequence. Expression or activity of native RecA machinery may be enhanced.

The integration may be via heterologous homologous recombination machinery. The heterologous recombination machinery may not be natively expressed by the organism. The vector may comprise a seventh genetic sequence encoding a heterologous recombination machinery. The heterologous recombination machinery may be comprised in a separate vector. The heterologous recombination machinery may be comprised in the target genome. Simultaneous expression of heterologous recombination machinery may facilitate homologous recombination.

### Positive Selection: Growing in the First Media and Selecting the Second Population

In some instances, the method comprises (b) growing the first population and the second population in a first media comprising a positive selection agent and a visual screening agent.

In some instances, the first media (i) permits growth of the second population, (ii) prevents growth of the first population, (iii) elicits the visual phenotype in the second population, and (iv) does not elicit the visual phenotype in the first population. The positive selection agent (i) may permit growth of the second population and (ii) may prevent growth of the first population. The first media may comprise agar and may be an agar plate. The positive selection agent may permit growth of the organism when the positive selection marker gene is present, and may prevent growth of the organism when the positive selection marker gene is not present. The visual screening agent (i) may elicit the visual phenotype in the second population and (ii) may not elicit the visual phenotype in the first population. The visual screening agent may elicit the visual phenotype when the reporter gene is present, and may not elicit the visual phenotype when the reporter gene is not present.

In some instances, the method comprises (c) selecting the second population based on a visual phenotype. The second population may be selected based on presence or absence of the visual phenotype. The visual phenotype may present when the visual screening agent is present. Presence of the visual phenotype at step (c) may indicate the vector successfully integrated into the second population. Thus, the second population may be selected by selecting a population of cells that grows in the presence of the positive selection agent and presents the visual phenotype.

Selection of the second population may be iterative (Isolation of clones on selection media or counter-selection media can be performed iteratively (e.g., colonies of the second population may be isolated on a media plate comprising the positive selection agent, then re-streaked onto another media plate comprising the positive selection agent).

### Excision and Counter-Selection: Growing m the Second Media and Generating the Third Population

In some instances, the method comprises (d) growing the second population in a second media comprising a first counter-selection condition, a second counter-selection condition, and the visual screening agent. The second media may not comprise the positive selection agent.

In some instances, the second media (i) permits growth of the third population, (ii) prevents growth of the second population, (iii) elicits the visual phenotype in the second population, and (iv) does not elicit the visual phenotype in the third population. The first counter-selection condition (i) may permit growth of the third population and (ii) may prevent growth of the second population. The second media may comprise agar and may be an agar plate. The first counter-selection condition may permit growth of the first population and the third population and may prevent growth of the second population. The first counter-selection condition may permit growth when the first counter-selection marker gene is present and may prevent growth when the first counter-selection marker gene is not present. The second counter-selection condition (i) may permit growth of the third population and (ii) may prevent growth of the second population. The second counter-selection condition may permit growth of the first population and the third population and may prevent growth of the second population. The second counter-selection condition may permit growth when the second counter-selection marker gene is present and may prevent growth when the second counter-selection marker gene is not present.

In some instances, growing the second population in the second media generates a third population of cells. In some instances, backbone of the vector may be excised from a target genome of the third population. Growing the second population in the second media may allow the backbone of the vector to excise from the target genomes of the second population, thus generating the third population.

The backbone may excise from the target genome of the second population upon absence of the positive selection agent, thus generating the third population. Excision of the vector backbone from the target genome of the third population may remove the vector backbone from the target genome of the third population. The target genome of the third population may retain the genetic modification sequence upon the excision. The excision is via homologous recombination. The excision is via heterologous homologous recombination machinery.

In some instances, the third population comprises regeneration of the wild-type organism. In some instances, the third population comprises the modified organism. The genetic modification sequence may knock out, disrupt, knock down, or inhibit activity of at least one sequence of interest or at least one gene product (e.g., a molecule, e.g., a protein) in the target genome of the third population. The genetic modification sequence may enhance expression or activity of at least one sequence of interest or at least one gene product in the target genome of the third population. The genetic modification sequence may introduce at least one sequence of interest to the target genome of the third population. The vector may produce a scarless and stable genetic modification in the target genome of the third population.

The systems and methods of the disclosure can be used with other suitable genetic engineering methods, for example, a CRISPR-Cas system. A CRISPR-Cas system may target degradation of a wild-type locus. A CRISPR-Cas system may select against the regeneration of a wild-type population following counter-selection.

### Selecting the Third Population and Screening for the Genetic Modification

In some instances, the method comprises (e) selecting the third population based on the visual phenotype. The visual phenotype may present when the visual screening agent is present. The third population may be selected based on presence or absence of the visual phenotype. Absence of the visual phenotype at step (e) may indicate either (i) the vector backbone excised from an organism after the vector successfully integrated into the organism to regenerate the wild-type genotype (e.g., a wild-type organism) or (ii) the vector backbone successfully excised from an organism after the vector successfully integrated into the organism to generate an intended mutation (e.g., a successfully modified organism). The visual screening agent may elicit the visual phenotype when the reporter gene is present, and may not elicit the visual phenotype when the reporter gene is not present. The visual screening agent (i) may elicit the visual phenotype in the second population and (iii) may not elicit the visual phenotype in the third population. Thus, successful excision of the vector backbone as expected for the modified organism may be selected by selecting a population of cells that grows in the presence of the counter-selection conditions and does not present the visual phenotype.

In some instances, the method comprises (f) screening the selected third population for a genetic modification sequence. The genetic modification sequence may be desired. The screening may facilitate selection of the successfully modified organism. The screening may be via PCR or sequencing. The screening may be via growing the selected third population in the presence of a selection agent or agents known to elicit phenotypic differences between the wild-type and modified organism. Since the vector backbone is excised from the third population of cells, growth of the selected third population in the presence of the positive selection agent or agents may indicate the vector backbone was not successfully excised.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Definitions

As used herein and in the appended claims, the singular forms “a,” “and,” and “the” include plural referents unless the context clearly dictates otherwise. Thus, for example, reference to “an agent” includes a plurality of such agents and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included.

The term “about” when referring to a number or a numerical range means that the number or numerical range referred to is an approximation within experimental variability (or within statistical experimental error), and thus the number or numerical range may vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The term “modification” with respect to a sequence or gene of interest or a nucleic acid (and related terms such as “modify” or “modified”) can mean mutation, knockout, truncation, knockdown, disruption, mitigation, inhibition, deletion, insertion, enhancement, or substitution. It can result in an increase or enhancement in expression of the modified gene. It can result in a decrease in expression of the modified gene.

### EXAMPLES

### Example 1: Method for Modifying and Selecting a Modified Organism

A modified organism may be prepared and selected according to the following protocol. The modification causes mutation, knockout, truncation, knockdown, disruption, mitigation, inhibition, deletion, insertion, enhancement, or substitution of a gene or sequence of interest in the genome of the modified organism.

First, electrocompetent cells are prepared by growing an organism in 25 mL of liquid media (5 g/L D-glucose, 10 g/L yeast extract, 10 g/L peptone, 3.38 g/L Na₂HPO₄·12 H₂O, 1.5 g/L citric acid, 20 mL/L ethanol, 10 mL/L acetic acid) at 30° C. shaking at 250 RPM in 50 mL baffled flasks until the organism reaches mid-log growth phase (OD₆₀₀~0.6-0.8).

The full culture of the electrocompetent cells and liquid media is then added to sterile 50 mL conical centrifuge tubes and held on ice for at least 10 minutes. The 50 mL conical centrifuge tubes are centrifuged at 5,000×G at 4° C. for at least 5 minutes. The supernatant is then removed from the 50 mL conical centrifuge tubes, leaving behind the cell pellet. The cell pellet is resuspended with 25 mL 1 mM sterile HEPES buffer, which is pre-chilled to 4° C. The 50 mL conical centrifuge tubes are then centrifuged at 5,000×G at 4° C. for at least 5 minutes. The supernatant is then removed from the 50 mL conical centrifuge tubes, leaving behind the cell pellet. The cell pellet is resuspended with 12.5 mL 1 mM sterile HEPES buffer, which is pre-chilled to 4° C. The 50 mL conical centrifuge tubes are then centrifuged at 5,000×G at 4° C. for at least 5 minutes. The supernatant is then removed from the 50 mL conical centrifuge tubes, leaving behind the cell pellet. The cell pellet is resuspended with about 1 mL (e.g., 0.8 mL) 1 mM sterile HEPES buffer, which is pre-chilled to 4° C.

Second, a non-replicating plasmid vector is introduced into the organism via electroporation. The vector can cause insertion, enhancement, deletion, or mitigation of a sequence of interest in the genome of the modified organism.

The vector comprises aphA1 as a positive selection marker gene, tetA as a first counter-selection marker gene, sacB as a second counter-selection marker gene, and lacZ as a reporter gene. aphA1 confers kanamycin resistance, sacB confers sucrose sensitivity, tetA confers both tetracycline resistance and fusaric acid sensitivity, and lacZ produces blue colonies in the presence of X-Gal. lacY facilitates transport of X-Gal into the cell. Homologous recombination between the vector and target genome either at a site upstream or downstream of the target modification yields clones that are selected using the positive selection marker (e.g., aphA1 and kanamycin or tetA and tetracycline resistance).

50-500 ng of the vector is added to 25 μL of the resuspended electrocompetent cells and transferred to a sterile 0.1 cm electroporation cuvette. The cuvette is electroporated at 1, 250 V, 25 μF, and 200Ω 1 mL of room temperature liquid media (5 g/L D-glucose, 15 g/L yeast extract, 3 g/L peptone) is then added to the cuvette to recover the electroporated cells. The electroporated cells are then incubated at 30° C., shaking at 250 RPM for at least 4 hours. 1 mL of the liquid culture is then centrifuged at 10,000×G for 1 minute. The supernatant is then removed, leaving behind a cell pellet. The cell pellet is resuspended in 100 μL of sterile 1 mM HEPES buffer.

Third, cells which have undergone a first recombination event (e.g., integration of the vector) undergo positive selection. The resuspended, electroporated cells are plated to a first media plate (e.g., a solid positive selection media (5 g/L D-glucose, 15 g/L yeast extract, 3 g/L peptone, 15 g/L agar, 75 ng/μL kanamycin, 20 mg/mL X-Gal)). The plated cells are then incubated at 30° C. for at least 48 hours or at least 72 hours. Blue colonies that appear on the first media plate are expected to contain bacteria that correctly encode the integrated vector sequence.

Fourth, cells which have undergone a double recombination event (e.g., integration of the vector and excision of the vector backbone via homologous recombination) are counter-selected (e.g., grown on plates containing sucrose and fusaric acid, owing to the first and second counter-selectable marker genes (e.g., sacB and tetA, respectively). The counter-selection step yields a mixture of cells encoding either a wild-type genotype or the desired modification. The theoretical yield of wild-type and mutant cells is 1:1, absent any fitness effect associated with the desired modification.

Kanamycin-resistant colonies are selected from the first media plate and streaked onto a second media plate (e.g., comprising a solid counter-selection media (15 g/L yeast extract, 3 g/L peptone, 200 g/L sucrose, 50 ng/μL fusaric acid, 20 mg/mL X-Gal)). The streaked cells are then incubated at 30° C. for at least 48 hours or at least 72 hours.

Fifth, cells which have undergone the double recombination event are screened for presence of a desired modification. Select white colonies that have grown on the solid counter-selection media are selected and screened (e.g., via PCR or sequencing) for presence of a desired modification.

### Example 2: Method for Modifying and Selecting a Modified Organism

A modified organism may be prepared and selected according to the protocol described in Example 1 using the vector in FIG. 1B. The modification causes truncation or mitigation of the wcaE gene in the genome of the modified organism, rendering the wcaE gene product (e.g., WcaE) non-active. Thus, the modification knocks out WcaE activity in the modified organism.

The modified organism is a bacteria. The bacteria is from the family Acetobacteraceae (e.g., an AAB). The Acetobacteraceae is a species from the genus Komagataeibacter. The Komagataeibacter is from the species Komagataeibacter europaeus. The Komagataeibacter europaeus is Komagataeibacter europaeus LMG 1521.

The vector introduced at step two comprises a wcaE coding sequence for truncation of a natively-expressed wcaE in the modified organism (FIG. 1B). At step three, the wcaE coding sequence, along with the rest of the vector, is integrated into the genome of the modified organism upon integration of the vector via homologous recombination. At step four, the wcaE coding sequence remains in the genome of the modified organism upon excision of the vector backbone. At step five, select white colonies that have grown on the solid counter-selection media are selected and screened for a truncated wcaE sequence.

### Example 3: Method for Modifying and Selecting a Modified Organism

A modified organism may be prepared and selected according to the protocol described in Example 1 using the vector in FIG. 1C. The modification causes introduction of a non-native sequence upstream of gqqA, resulting in the constitutive overexpression of the gqqA gene. Thus, the modification increased GqqA activity in the modified organism.

The modified organism is a bacteria. The bacteria is from the family Acetobacteraceae (e.g., an AAB). The Acetobacteraceae is a species from the genus Komagataeibacter. The Komagataeibacter is from the species Komagataeibacter europaeus. The Komagataeibacter europaeus is Komagataeibacter europaeus LMG 1521.

The vector introduced at step two comprises a tac promoter sequence operationally linked to an optimized RBS sequence operationally linked to the native gqqA coding sequence in the modified organism (FIG. 1C). At step three, tac promoter sequence operationally linked to an optimized RBS sequence operationally linked to the native gqqA coding sequence, along with the rest of the vector, is integrated into the genome of the modified organism upon integration of the vector via homologous recombination. At step four, the tac promoter sequence operationally linked to an optimized RBS sequence operationally linked to the native gqqA coding sequence remains in the genome of the modified organism upon excision of the vector backbone. At step five, white colonies that have grown on the solid counter-selection media are selected and screened for the tac promoter sequence operationally linked to an optimized RBS sequence operationally linked to the native gqqA coding sequence.

1. A vector comprising:
a first nucleic acid sequence comprising a positive selection marker gene,
a second nucleic acid sequence comprising a first counter-selection marker gene,
a third nucleic acid sequence comprising a second counter-selection marker gene, and
a fourth nucleic acid sequence comprising a reporter gene.

2. The vector of claim 1, comprising:
a fifth nucleic acid sequence comprising a first homology arm that comprises homology with a first portion of a target genomic sequence of an organism targeted for modification, and
a sixth nucleic acid sequence comprising a second homology arm that comprises homology with a second portion of said target genomic sequence of said organism targeted for modification.

3. (canceled)

4. The vector of claim 1, wherein said positive selection marker gene encodes aminoglycoside phosphotransferase, a class A tetracycline resistance protein, chloramphenicol acetyltransferase, or beta-lactamase.

5. (canceled)

6. The vector of claim 1, wherein said positive selection marker gene encodes orotate phosphoribosyl-transferase (pyrE) galactokinase 1 (galK), thymidylate synthase (thyA), orotidine-5′-monophosphate decarboxylase (pyrF), or uracil phosphoribosyltransferase (upp).

7. (canceled)

8. The vector of claim 1, wherein said first counter-selection marker gene encodes levansucrase, a class A tetracycline resistance protein, uracil phosphoribosyl-transferase, orotate phosphoribosyl-transferase, or cytosine deaminase.

9. The vector of claim 8, wherein said second counter-selection marker gene encodes levansucrase, a class A tetracycline resistance protein, uracil phosphoribosyl-transferase, orotate phosphoribosyl-transferase, or cytosine deaminase.

10. (canceled)

11. The vector of claim 1, wherein said reporter gene comprises lacZ, a lacZ homolog, a gene encoding green fluorescent protein (GFP), a gene encoding red fluorescent protein (RFP), or a gene encoding luciferase (luc).

12. (canceled)

13. The vector of claim 2, wherein any of said first, second, third, fourth, fifth, or sixth nucleic acid sequences are operably linked to a regulatory element.

14. The vector of claim 13, further comprising one or more of a promoter, a ribosomal binding site (RBS), a transcription terminator, an origin of transference (OriT), an origin of replication (Ori), a multiple cloning site (MCS), or a transporter.

15-27. (canceled)

28. A system for genetic modification of an organism comprising said vector of claim 2.

29. The system of claim 28, wherein said positive selection marker gene (i) confers resistance to an antibiotic or (ii) creates a cell survival molecule.

30. (canceled)

31. The system of claim 29, wherein said antibiotic is kanamycin, tetracycline, chloramphenicol, ampicillin, carbenicillin, gentamicin, or erythromycin.

32. (canceled)

33. The system of claim 29, wherein said cell survival molecule is orotate phosphoribosyl-transferase, galactokinase 1, thymidylate synthase, orotidine-5′-monophosphate decarboxylase, or uracil phosphoribosyltransferase.

34-45. (canceled)

46. A method for modifying an organism, comprising:
introducing said vector of claim 1 to said organism, wherein introducing said vector to said organism causes a genetic modification in said organism to produce a modified organism.

47. A method for selecting a modified organism, comprising:
introducing said vector of claim 1 to a first population of cells, wherein introducing said vector to said first population generates a second population of cells, wherein said vector is integrated into a target genome of said second population;
growing said first population and said second population in a first media comprising a positive selection agent and a visual screening agent;
selecting said second population based on a visual phenotype;
growing said second population in a second media comprising a first counter-selection condition, a second counter-selection condition, and said visual screening agent, wherein growing said second population in said second media generates a third population of cells, wherein a backbone of said vector is excised from a target genome of said third population, and wherein said third population comprises said modified organism; and
selecting said third population based on said visual phenotype.

48. A method for selecting a modified organism, comprising:
introducing a vector to a first population of cells, wherein introducing said vector to said first population generates a second population of cells, and wherein said vector is integrated into a target genome of said second population;
growing said first population and said second population in a first media comprising a positive selection agent and a visual screening agent;
selecting said second population based on a visual phenotype;
growing said second population in a second media comprising a first counter-selection condition, a second counter-selection condition, and said visual screening agent, wherein growing said second population in said second media generates a third population of cells, wherein a backbone of said vector is excised from a target genome of said third population, and wherein said third population comprises said modified organism; and
selecting said third population based on said visual phenotype.

49. (canceled)

50. The method of claim 48, wherein the vector comprises a first nucleic acid sequence comprising a positive selection marker gene, a second nucleic acid sequence comprising a first counter-selection marker gene, a third nucleic acid sequence comprising a second counter-selection marker gene, and a fourth nucleic acid sequence comprising a reporter gene.

51-56. (canceled)

57. The method of claim 48, wherein said positive selection agent (i) permits growth of said second population and (ii) prevents growth of said first population.

58. (canceled)

59. (canceled)

60. The method of claim 48, wherein said visual screening agent (i) elicits said visual phenotype in said second population and (ii) does not elicit said visual phenotype in said first population.

61-66. (canceled)

67. The method of claim 48, wherein said first counter-selection condition (i) permits growth of said third population and (ii) prevents growth of said second population.

68-71. (canceled)

72. The method of claim 48, wherein said second counter-selection condition (i) permits growth of said third population and (ii) prevents growth of said second population.

73-76. (canceled)

77. The method of claim 48, wherein excision of said vector backbone from said target genome of said third population removes said vector backbone from said target genome of third second population.

78-83. (canceled)

84. The method of claim 48, wherein said vector produces a scarless and stable genetic modification in said target genome of said third population.

85-88. (canceled)

89. A method for selecting a modified organism, comprising:
introducing a vector to a first population of cells,
wherein introducing said vector to said first population generates a second population of cells, and
wherein said vector is integrated into a target genome of said second population, and
wherein said vector introduces a genetic modification sequence to said target genome of said second population;
growing said first population and said second population in a first media comprising a positive selection agent and a visual screening agent,
wherein said positive selection agent (i) permits growth of said second population and (ii) prevents growth of said first population, and
wherein said visual screening agent (i) elicits a visual phenotype in said second population and (ii) does not elicit said visual phenotype in said first population;
selecting said second population based on said visual phenotype;
growing said second population in a second media,
wherein said second media does not comprise said positive selection agent, wherein said second media comprises a first counter-selection condition, a second counter-selection condition, and said visual screening agent,
wherein said first counter-selection condition (i) permits growth of said third population and (ii) prevents growth of said second population,
wherein said second counter-selection condition (i) permits growth of said third population and (ii) prevents growth of said second population,
wherein said visual screening agent (i) elicits said visual phenotype in said second population and (iii) does not elicit said visual phenotype in said third population,
wherein growing said second population in said second media generates a third population of cells,
wherein a backbone of said vector is excised from a target genome of said third population,
wherein said target genome of said third population comprises said genetic modification sequence, and
wherein said third population comprises said modified organism;
selecting said third population based on said visual phenotype; and
screening said selected third population for said genetic modification sequence.
